(19) United States
(12) Patent Application Publication
(10) Pub. No.: US 2011/0060236 A1
(43) Pub. Date: Mar. 10, 2011

(54) Method for Measuring Breath Temperature in Breath Alcohol Testing

(75) Inventors: Hans-Olov Moberg, Bro (SE); Lars-Å Lejon, Uppsala (SE)

(73) Assignee: NANOPULS AB, Uppsala (SE)

(21) Appl. No.: 12/293,753

(22) PCT Filed: Mar. 7, 2007
(86) PCT No.: PCT/SE07/00218

§ 371 Date: Sep. 19, 2008

(30) Foreign Application Priority Data
Mar. 31, 2006 (SE) 0600724-9

(51) Int. Cl. A61B 5/08

(52) U.S. Cl. 600/529

(57) ABSTRACT

In a method for determining ethanol concentration by exhalation tests there is obtained an additional feature in indirect measuring of the temperature of the exhalation air by making an IR measurement at a wave length with a high absorption of water molecules.

### FIELD OF THE INVENTION

The present invention pertains to the field of breath alcohol testing based on the IR technique, i.e. the absorbance of IR energy by ethanol molecules in the micrometer range. More specifically the invention pertains to a method for measuring breath temperature of the person being tested. The breath temperature could be used as an indirect indication of the body temperature which has an influence on alcohol measurement results.

### BACKGROUND OF THE INVENTION

Alcohol exhalation tests are commonly used e.g. at road side traffic controls. In certain countries however such tests until recently only served the purpose of giving a preliminary indication of the possible unpermitted alcohol content of the blood of the person being tested. Confirmatory tests usable for law enforcement had to be made in a stationary clinic or police station which was costly, time-consuming and involved a risk that the metabolism brought the alcohol levels below permitted levels before the confirmatory test was made.

However, there is now available mobile testing equipment enabling tests of a quality and standard usable for law enforcement. An example of such a system is the EVIDENZER system, manufactured and sold by NANOPULS AB, Uppsala, Sweden. This system is based on the well-established reliable IR technique. Five narrow bands precision ER filters in the range of 3.3-3.8 μm are used to measure the ethanol concentration in the breath.

However, it is also a well-known fact that the concentration of ethanol increases with increasing temperature. Normally the breath temperature of a healthy person holds a temperature of about 34° C. and it is estimated that an increase of this temperature will increase the ethanol concentration of about 6-7 percent per degree. As the rules for law enforcement are normally related to blood concentrations of ethanol, a feverish person might erroneously pass the level of permissible alcohol concentration. In order to compensate for this artifact it has been suggested in IR measurement of breath alcohol testing to provide the equipment with an additional separate measuring device, such as a thermistor, at the mouth-piece of the equipment for directly measuring the breath temperature. However, this makes the measurement results sensitive to temperature variations of the mouth-piece.

### SUMMARY OF THE INVENTION

It is therefore an object of the present invention to provide a method and a device for a simple and cost effective indirect measurement of the breath temperature in IR breath alcohol testing.

The characteristics of the invention will appear from the enclosed claims.

### DESCRIPTION OF THE INVENTION

The invention will now be described in detail, reference being made to the enclosed drawing in which:

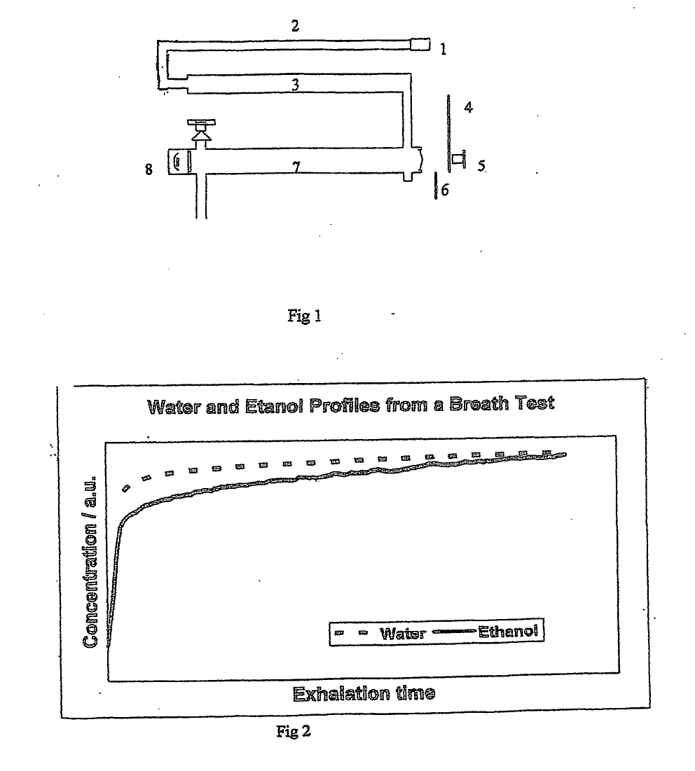

FIG. 1 is a schematic view of an IR breath alcohol testing device modified so as to imply the invention and

FIG. 2 is a diagram showing the relation between the concentration of water and ethanol vs time in exhalation air as measured in accordance with the invention.

In FIG. 1, which is a simplified schematic sketch of an IR breath alcohol testing device, reference 1 denotes a mouth-piece, reference 2 denotes a breath hose, reference 3 denotes a preheater, reference 4 denotes a filter wheel, reference 5 denotes a detector, reference 6 denotes a reference filter, reference 7 denotes a measuring chamber and reference 8 denotes an IR-source. Typically in the equipment known per se the filter wheel would include four different measurement filters in the wave lengths range 3.3-3.8 μm generating four different output signals from the IR-source in the detector. These signals together form an adequate indication of the ethanol contents of the air from the breath filling the measuring chamber. This is e.g. the case in the above mentioned EVIDENZER system which is hereby by reference incorporated as part of the specification.

According to the present invention there is included in the filter wheel 4 an additional fifth measuring band pass filter having a wave length at which water vapor has a high absorbance. A suggested value would be 3,05 μm but any wavelength at which water has a high absorption can be used with a suitable detector. Preferably a wavelength should be chosen where any possible disturbing substances have a low absorption.

FIG. 2 is a diagram showing the relation between the concentration of water and ethanol vs time in exhalation air as measured by the equipment according to FIG. 1. If the temperature of the exhalation air increases the curve for water will move almost linearly upwards with temperature and thus a measurement of breath temperature and indirectly a measurement of body temperature could be made by only making a minor modification of the existing equipment.

Thus, by simply introducing an additional filter in the filter wheel an additional measurement signal will be generated representing the temperature of the breath of the person being subject to test and thus an indirect indication of the body temperature of the tested person will be obtained. This value could then be used for recalculation the ethanol levels measured if desired.

1.-2. (canceled)

3. A method in alcohol breath testing using infrared (IR) absorption of ethanol molecules for additionally indirectly measuring a breath temperature of a person tested, comprising in addition to measuring an IR absorption of ethanol, measuring an IR absorption of water.

4. The method of claim 3, wherein the IR absorption of water is measured at a wave length of 3.05 micrometers.
